(19) United States
(12) Patent Application Publication
(10) Pub. No.: US 2025/0043334 A1
(43) Pub. Date: Feb. 6, 2025

(54) COLD-TEMPERATURE ISOTHERMAL AMPLIFICATION OF POLYNUCLEOTIDES

(71) Applicant: Massachusetts Institute of Technology, Cambridge, MA (US)

(72) Inventors: Nicolaas Angenent-Mari, Somerville, MA (US); James j. Collins, Newton, MA (US)

(73) Assignee: Massachusetts Institute of Technology, Cambridge, MA (US)

(21) Appl. No.: 18/714,546

(22) PCT Filed: Dec. 7, 2022
(86) PCT No.: PCT/US2022/081085

§ 371 Date: May 29, 2024

Related U.S. Application Data
(60) Provisional application No. 63/287,437, filed on Dec. 8, 2021.

(51) Int. Cl. C12Q 1/6818; C12Q 1/682

(52) U.S. Cl. CPC C12Q 1/6818; C12Q 1/682

(57) ABSTRACT

In some aspects, the present disclosure provides methods and compositions for isothermal low temperature amplification of target polynucleotides, and detection thereof.

### RELATED APPLICATION

This application claims the benefit under 35 U.S.C. § 119 (c) of U.S. provisional application No. 63/287,437, filed Dec. 8, 2021, which is incorporated by reference herein in its entirety.

### REFERENCE TO AN ELECTRONIC SEQUENCE LISTING

The contents of the electronic sequence listing (H049870749WO00-SEQ-HJD.xml; Size: 7,196 bytes; and Date of Creation: Dec. 2, 2022) is herein incorporated by reference in its entirety.

### BACKGROUND

The need for at-home point-of-care (POC) nucleic acid testing products (NATs) that can be marketed directly to the consumer has become increasingly apparent due to the ongoing SARS-COV-2 pandemic. While a handful of such products taking advantage of advances in NAT-related technologies have reached the market, key improvements over available underlying technologies are needed.

### SUMMARY

Provided herein, in some aspects, are compositions and methods for isothermal amplification of target polynucleotides that are robust at low temperatures (e.g., 12-25° C.). Low temperature isothermal amplification of target polynucleotides is an important step towards producing low cost, at home tests for detecting target polynucleotides associated with a disease, for example, COVID-19, cause by SARS-COV-2 infection. Low temperature target polynucleotide amplification is achieved, at least in part, using primers specifically designed to inhibit primer dimer formation at low temperatures.

The primers provided herein, in some embodiments, comprise a short (e.g., 10-30 nucleotides) hybridization sequence that is complementary to a primer binding sequence on the target polynucleotide. The hybridization sequence, in some embodiments, further comprises a nickase recognition sequence (e.g., a partial or complete nickase recognition sequence). In some embodiments, the primer also comprises a stabilization sequence that is 5′ to the nickase recognition sequence. In some embodiments, the primer further comprises a 3′ blocking molecule that terminates polymerization, thereby blocking amplification of primer dimers. In some embodiments, the 3′ blocking molecule is later removed from the primer using a nickase and strand displacing polymerase to enable 3′ elongation of the primer as explained below.

The target polynucleotide to be amplified and detected, in some embodiments, comprises (e.g., is modified to comprise) a primer binding sequence located near (e.g., within 50 nucleotides) of the 5′ end and/or a primer binding sequence located near (e.g., within 50 nucleotides) of the 3′ end of the target polynucleotide. In some embodiments, the target polynucleotide to be amplified and detected comprises (e.g., is modified to comprise) a primer binding sequence located at the 3′ end of the target polynucleotide. In some embodiments, a target polynucleotide primer binding sequence comprises a nickase recognition sequence, which may be a partial nickase recognition sequence or complete nickase recognition sequence.

One example of the methods provided herein is depicted in FIGS. 1A-1J. Without being bound by theory, a primer first binds to the primer binding sequence(s) of the target polynucleotide. For example, the primer may bind to a primer binding sequence on the target polynucleotide that comprises a nickase recognition sequence (FIG. 1A). In some embodiments, a primer binds to a primer binding sequence within 50 nucleotides of the 5′ end of the target polynucleotide, and a strand displacing polymerase adds the reverse complement of the primer to the target polynucleotide by elongating in the 3′to 5′ direction to (FIG. 1B). A nickase, in some embodiments, then binds the newly formed double stranded target polynucleotide and nicks the primer, which enables the strand displacing polymerase to remove the 3′ blocking molecule and elongate the primer (FIG. 1C-1D). Elongation of the remainder of the target polynucleotide forms a double stranded target polynucleotide comprising a primer stabilization sequence and a nickase recognition sequence (FIG. 1E). Target polynucleotide amplification may continue through repeated cycles of nicking and elongation. Single stranded DNA binding proteins are added, in some embodiments, to stabilize the displaced strand during elongation (FIG. 1F). In some embodiments, repeated nicking and elongation results in repeated production of the reverse complement of the target polynucleotide (FIG. 1G). In some embodiments, the primer also binds to the reverse complement of the target polynucleotide (FIG. 1H). In some embodiments, a second primer binds to the reverse complement of the target polynucleotide. This results in nicking and elongation of the reverse complement copies of the target polynucleotide, which produces copies of the target polynucleotide (FIGS. 1I-1J). This in turn results exponential amplification of the target polynucleotide. In some embodiments, the copies of the target polynucleotide (or the reverse complement of the target polynucleotide) that are produced by amplification are detected, for example, using the detection methods described herein. Unlike previous techniques this amplification and detection scheme is robust in isothermal conditions at low temperatures.

Some aspects of the present disclosure provide a composition comprising: (a) a target polynucleotide comprising a primer binding sequence, wherein the primer binding sequence comprises a nickase recognition sequence; and (b) a primer comprising a hybridization sequence complementary to the primer binding sequence, wherein (i) the primer further comprises a 3′ blocking molecule and/or (ii) the hybridization sequence consists of 8-12 contiguous nucleotides complementary to the primer binding sequence.

In some embodiments, the target polynucleotide is single stranded.

In some embodiments, the nickase recognition sequence is a partial or complete nickase recognition sequence.

In some embodiments, the primer binding sequence is in a 5′ end region of the target polynucleotide.

In some embodiments, rein the target polynucleotide comprises an additional sequence that is a reverse complement to the primer binding sequence.

In some embodiments, the additional sequence is in a 3′ end region of the target polynucleotide.

In some embodiments, the 3′ blocking molecule is selected from 3′ hexanediol, 3′ddC. 3′ Inverted dT, 3′ carbon chain spacer, 3′ amino, and 3′ phosphorylation.

In some embodiments, the primer further comprises a stabilization sequence 5′ to the nickase recognition sequence, optionally wherein the stabilization sequence consists of 6-30 nucleotides or 18 nucleotides.

In some embodiments, the target polynucleotide is present in the composition at a concentration of less than 100 attomolar.

In some embodiments, the composition further comprises a cognate nickase, a single-strand binding protein, a strand displacing polymerase, or any combination thereof.

In some embodiments, the cognate nickase is selected from Nb.BbvCI, Nt.BspQI, Nt.CviPII, Nt.BstNBI, Nb.BsrDI, Nb.BtsI, Nt.AlwI, Nt.BbvCI, Nb.BsmI, Nb.BssSI, and Nt.BsmAI.

In some embodiments, the single-strand binding protein is selected from T4 Gene 32 Protein (T4gp32), Tth RecA, and ET SSB.

In some embodiments, the strand displacing polymerase is selected from Bsu DNA Polymerase I (Bsu), phi29, Bst DNA Polymerase, Klenow Large Fragment, Klenow Exo-, Bsu Large Fragment, Isopol, and Isopol SD+.

In some embodiments, the composition further comprises (i) a Cas13a or Cas13b protein, (ii) a detector polynucleotide comprising a sequence flanked by a detectable molecule and a quencher molecule, (iii) a crRNA, and (iv) an RNA polymerase, wherein the target polynucleotide further comprises a cognate RNA polymerase binding sequence, and wherein the crRNA comprises a sequence that binds to RNA transcribed from the target polynucleotide. Other aspects of the present disclosure provide a method comprising incubating a composition provided herein in a buffer to produce multiple copies of the target polynucleotide.

In some embodiments, the incubating is between about 4 degrees Celsius to about 50 degrees Celsius, about 4 degrees Celsius to about 45 degrees Celsius, about 4 degrees Celsius to about 40 degrees Celsius, about 4 degrees Celsius to about 35 degrees Celsius, about 4 degrees Celsius to about 30 degrees Celsius, or about 16 degrees Celsius to about 25 degrees Celsius.

In some embodiments, the incubating is in isothermal conditions.

In some embodiments, the method further comprises detecting the multiple copies of the target polynucleotide, optionally wherein the detecting uses specific high-sensitivity enzymatic reporter unlocking (SHERLOCK).

Yet other aspects of the present disclosure provide a method comprising incubating a composition provided herein in a buffer to produce multiple copies of the target polynucleotides, and then incubating the multiple copies of the target polynucleotide with a Cas 12a protein, a crRNA, and a detector polynucleotide comprising a detector sequence flanked by a detectable molecule and a quencher molecule, wherein the crRNA comprises a sequence that binds to the target polynucleotide or the reverse complement of the target polynucleotide.

In some embodiments, the method further comprises detecting the multiple copies of the target polynucleotide, optionally wherein the detecting uses specific high-sensitivity enzymatic reporter unlocking (SHERLOCK).

### BRIEF DESCRIPTION OF DRAWINGS

FIGS. 1A-1J show a schematic for target oligonucleotide amplification. FIG. 1A shows a schematic of a primer comprising a 3′ blocking molecule. FIG. 1B shows elongation of the target oligonucleotide using the primer as a template and BSU. FIG. 1C show the primer being nicked by the Nickase. FIG. 1D show Bsu elongating down the primer using the target polynucleotide as a template and displacing the nicked portion of primer comprising the 3′ blocking group. FIG. 1E shows nicking of the double stranded target polynucleotide product from FIG. 1D. FIG. 1F shows amplification of the double stranded target polynucleotide product using BSU and stabilization of the displaced strand with a single-stranded binding protein (SSBP). FIG. 1G shows the results of repeated amplification from the double stranded target polynucleotide product. FIG. 1H shows the primer comprising a 3′ blocking molecule binding to the single stranded reverse complement of the target polynucleotide. FIG. 1I shows nicking of the double stranded DNA produced by elongation reverse complement of the target polynucleotide produced in FIG. 1H. FIG. 1J shows repeated nicking and elongation of the reverse complement of the target polynucleotide.

FIG. 2 shows that the CRISPR-Cas9-triggered nicking endonuclease-mediated strand displacement amplification (CRISDA) platform is not robust at lower temperatures. CRISDA is current state of the art ambient temperature stand displacement amplification (SDA) using existing primer designs. Amplification reactions identical to those described in the CRISDA publication (Zhou, Wenhua, et al. Nature Communications, vol. 9, no. 1, 2018) were carried out with primer length, primer concentration, and single-strand binding protein (T4gp32) concentration varied as indicated. Fold-change in signal from a second-step Cas 12a detection reaction is shown at different concentrations of an input ssDNA trigger (0 attomolar (aM), 20aM, 2fM, 200fM).

FIG. 3 shows that decreasing primer length decreases production of primer dimers. Time course of strand displacement amplification (SDA) primer dimer formation at room temperature. Agarose gels (4%) of SDA reactions stopped at indicated timepoints (by heat killing at 80° C.) are shown for three pairs of primers of varying length (20 nucleotides (nt), 17nt and 14nt). Red arrows and red text indicate the timing of formation of nickase-extended products smaller than the initial starting primers.

FIGS. 4A-4B shows a new primer design with a short hybridization sequence of 9nt. FIG. 4A Left: Primers are designed to dimerize such that the nickase recognition sequence (BbvCI) does not completely overlap. Right: Primers that dimerize such that the nickase recognition sequence is excluded from the hybridization sequence will produce nickase recognition sequences and an exponentially self-amplifying dimer species. FIG. 4B previous designs for strand displacement amplification employ two primers with hybridization sequences (black) as well as nickase recognition sequences (green) and stabilization sequences (pink). These primers are long enough to allow for the formation of exponentially amplifying dimer species. However, the improved short single primer design described herein form only inert non-amplifying dimer species. This is made possible by binding primer binding sequences (blue) onto the 3′ end and 5′ end of the target polynucleotide. In some embodiments, the primer binding sequences at the 3′ end and the 5′ end of the target polynucleotide are identical sequences. In some embodiments, the primer binding sequences at the 3′ end and the 5′ end of the target polynucleotide are different sequences. The nickase recognition sequence (green) can also be added at this time. The primer binding sequence and nickase recognition sequence may be added to the target polynucleotide by a variety of means, including the use of bump primers, ligase splints, etc. The removal of the target-specific region and the consolidation of two primers into one allows for short primers to be used in a way that is not otherwise possible.

FIG. 5 Suggests the short primer design does not result in exponential primer dimer amplification. Time course primer dimer formation at room temperature using new primer design. Agarose gels (4%) of reactions stopped at indicated timepoints (by heat killing at 80° C.) are shown for a single shortened 9nt forward primer. Red text indicates the timing of formation of primer dimer, which is notably not nickase-generated (such a product would be smaller than the starting primer rather than dramatically larger).

FIGS. 6A-6B show ambient-temperature detection of single-stranded DNA (ssDNA) target polynucleotides using Cas12a and short primers. FIG. 6A shows a two pot reaction schematic for amplification and detection of target polynucleotide using Cas12a. An ssDNA target polynucleotide was amplified for two hours at room temperature using short primers. After amplification, a crRNA: Cas12a complex targeting a PAM-containing sequence on the amplicon was added to the reactions and allowed to cleave a fluorophore-quencher reporter (e.g., a detector polynucleotide) for thirty minutes. FIG. 6B shows quantification of detection for varying amounts of starting ssDNA target polynucleotide. Individual values and mean of six replicates are shown (n=6).

FIGS. 7A-7C show ambient-temperature detection of ssDNA target polynucleotides using Cas 13a and amplification with short primers. FIG. 7A shows one pot amplification and detection of ssDNA target polynucleotides using Cas13a. An ssDNA target polynucleotide was amplified at room temperature using the primers and addition of T7 RNA polymerase (RNAP) to the system. During amplification, a crRNA: Cas13a complex present in the reaction detected transcribed RNA copies of the target polynucleotide and in response cleaved a fluorophore-quencher reporter (i.e., a detector polynucleotide). Actual reaction start time was 45 minutes before t=0. FIG. 7B shows detection of copies of the target polynucleotide over time for varying amounts of starting target polynucleotide. FIG. 7C shows the temperature sensitivity of the assay between 20° C. and 12° C. Individual time courses of four replicates are shown (n=4).

FIG. 8 shows ambient-temperature detection of single stranded target polynucleotide using Cas 13a and primers with 3′ blocking molecules. A single stranded target polynucleotide was amplified at room temperature using short primers with either an unblocked 3′ OH, or a 3′ hexanediol blocking molecule, and in the presence or absence of background single stranded DNA (ssDNA) oligos (25 nM). During amplification, a crRNA: Cas13a complex present in the reaction detected transcribed RNA copies of the target polynucleotide, and in response cleaved a detector polynucleotide. Actual reaction start time was 80 minutes before setting the reaction on the plate reader. Individual time courses of single replicates are shown (n=1).

FIG. 9 shows that different strand displacing polymerases can be used to detect femtomolar (fM) amounts of target polynucleotide. Amplified copies of the target polynucleotide were detected using an enzyme fluorophore quencher assay. The reaction was performed with a short 3′ blocked primer (10nt hybridization sequence, 18nt stabilization sequence), at ambient temperature, with a T7 transcription step followed by Cas13 fluorescence detection in a one-pot reaction.

### DETAILED DESCRIPTION

In some aspects, the present disclosure relates compositions and methods for isothermal, low temperature amplification of a target polynucleotide. In some embodiments, amplification is followed by detection of copies of the target polynucleotide.

### Compositions

Provided herein, in some aspects, are compositions that may be used to amplify a target polynucleotide, for example, at ambient temperature in isothermal conditions. The compositions, in some embodiments, include a target polynucleotide and primers designed to bind specifically to the target polynucleotide (e.g., flanking a sequence of interest). The compositions, in some embodiments, also include a nickase, a single-strand binding protein, and/or a strand displacing polymerase, each of which is described below.

In some embodiments, molecules for detection of the amplified target polynucleotide are also included or added to the composition. For example, a composition may further comprise a detector polynucleotide flanked by a detectable molecule and a quencher molecule. In some embodiments, the detector polynucleotide includes an endonuclease recognition sequence. The detector polynucleotide, in some embodiments, is included in a composition along with a Cas 12 endonuclease, or alternatively, with a Cas13 endonuclease and an RNA polymerase. The detector polynucleotide, in some embodiments, is cleaved by collateral activity of the Cas12 endonuclease or Cas13 endonuclease as described in Kellner et al. Nature protocols 14.10 (2019): 2986-3012, which is incorporated by reference in its entirety.

### Target Polynucleotide

The methods of the present disclosure are used to amplify a target polynucleotide. The target polynucleotide may be DNA or RNA, or a hybrid of DNA and RNA. In some embodiments, the target polynucleotide is an RNA, for example, an mRNA, a non-coding RNA, or a ribosomal RNA. In some embodiments, the target polynucleotide is single stranded. In other embodiments, the target polynucleotide is double stranded. In yet other embodiments, the target polynucleotide is partially double stranded (i.e., includes a single-stranded region and a double-stranded region).

In some embodiments, the target polynucleotide is from a pathogen. A pathogen is generally known to be a bacterium, a virus, or other microorganism that can cause disease. In some embodiments, the target polynucleotide is a viral polynucleotide. For example, the target polynucleotide may be from a virus, such as a betacoronavirus, one non-limiting example of which is SARS-COV-2. In other embodiments, the target polynucleotide is a bacterial polynucleotide. In yet other embodiments, the target polynucleotide is a fungal polynucleotide. In yet other embodiments, the target polynucleotide is a protist or parasitic polynucleotide. In some embodiments, the target polynucleotide is unique to a pathogen such that detection of the target polynucleotide indicates the presence of a specific pathogen.

In some embodiments, the target polynucleotide is present in a sample from (e.g., obtained from) a subject. A “subject” includes, but is not limited to, humans and other non-human animals including, for example: companion animals such as dogs, cats, domesticated pigs, ferrets, and hamsters; primates such as cynomolgus monkeys and rhesus monkeys; and agricultural animals such as cattle, pigs, horses, sheep, goats, and birds (e.g., chickens, ducks, geese, and/or turkeys). In some embodiments, the subject is a human subject.

The sample, in some embodiments, is selected from blood, urine, saliva, and mucus. In some embodiments, the sample is a blood sample. In other embodiments, the sample is a urine sample. In yet other embodiments, the sample is a saliva sample. In still other embodiments, the sample is a mucus sample.

In some embodiments, the compositions and methods disclosed herein result in robust amplification of the target polynucleotide. Robust amplification of a target polynucleotide refers to compositions and methods that consistently amplify the target polynucleotide to a detectable level. Consistent amplification is evidenced by, for example, repeatable success in amplifying and detecting the target polynucleotide, as exemplified in Example 2. A person of skill will understand that robustness is dependent on the starting concentration of target polynucleotide in the composition or method. For example, the compositions and methods described herein are generally expected to robustly amplify and detect attomolar amounts of a target polynucleotide e.g., at least 2 aM, at least 3 aM, at least 4 aM, at least 5 aM, at least 10 aM, at least 20 aM, at least 50 aM, or at least 100 aM of the target polynucleotide.

In some embodiments, the target polynucleotide is present in the sample at a concentration of at least 2 attomolar (aM). For example, the target polynucleotide may be present in the sample at a concentration of at least 2 aM, at least 3 aM, at least 4 aM, at least 5 aM, at least 10 aM, at least 20 aM, at least 50 aM, or at least 100 aM. In some embodiments, the target polynucleotide is present in the sample at a concentration of 2-5 aM, 2-10 aM, 2-20 aM, 2-40 aM, 2-100 aM, 5-10 aM, 5-20 aM, 5-40 aM, 5-100 aM, 10-20 aM, 10-50 aM, 20-50 aM, 10-100 aM, 50-100 aM, 1-1000 aM, 5-1000aM, or 50-1000 aM. In some embodiments, the sample comprises other molecules in addition to the target polynucleotide, for example, other non-target polynucleotides.

### Primer Binding Sequences

In some embodiments, the target polynucleotide comprises one or more primer binding sequences. For example, the target polynucleotide may comprise a first primer binding sequence and a reverse complement of a second primer binding sequence, wherein the first primer binding sequence and the reverse complement of the second primer binding sequences flank a sequence of interest. A sequence of interest refers to a sequence within the target polynucleotide that is specifically amplified. This sequence may comprise a sequence that is bound by a CRISPR Cas complex during detection. The first primer binding sequence and the reverse complement of a second primer binding sequence are considered to “flank” a sequence of interest when one primer binding sequence (or reverse complement thereof) is upstream from (5′ to) the sequence of interest, and the other primer binding sequence (or reverse complement thereof) is downstream from (3′ to) the sequence of interest. This configuration enables polymerase-based amplification of the intervening sequence of interest. It should be understood that embodiments describing a “primer binding sequence” encompass a “first primer binding sequence,” a “reverse complement of a second primer binding sequence,” and any additional primer binding sequences that may be contemplated.

In some embodiments, a primer binding sequence comprises 8-12 contiguous nucleotides. Contiguous nucleotides are nucleotides consecutive in the primary sequence of the target polynucleotide with no additional intervening nucleotides or other molecules between the consecutive nucleotides. In some embodiments, a primer binding sequence comprises 8-9, 8-10, 8-11, 8-12, 9-10, 9-11, 9-12, 10-11, 10-12, or 11-12 contiguous nucleotides. In some embodiments, a primer binding sequence comprises 9-11 contiguous nucleotides. In some embodiments, a primer binding sequence comprises 7, 8, 9, 10, 11, 12, 13, 14, 15, 16, 17, 18, 19, 20, 21, 22, 23, 24, 25, 26, 27, 28, 29, 30, or more contiguous nucleotides. In some embodiments, a primer binding sequence comprises 8-13, 8-14, 8-15, 8-16, 8-17, 8-18, 8-19, 8-20, 8-21, 8-22, 8-23, 8-24, 8-25, 8-26, 8-27, 8-28, 8-29, or 8-30 contiguous nucleotides. In some embodiments, a primer binding sequence comprises 8 contiguous nucleotides. In some embodiments, a primer binding sequence comprises 9 contiguous nucleotides. In some embodiments, a primer binding sequence comprises 10 contiguous nucleotides. In some embodiments, a primer binding sequence comprises 11 contiguous nucleotides. In some embodiments, a primer binding sequence comprises 12 contiguous nucleotides.

In some embodiments, a primer binding sequence is attached to the 5′ terminal nucleotide of the target polynucleotide. In some embodiments, a primer binding sequence is located within the 5′ end region of the target polynucleotide. In some embodiments, the 5′ end region of the target polynucleotide refers to the 7, 8, 9, 10, 11, 12, or 13 most terminal nucleotides on the 5′ end. In some embodiments, the 5′ end region of the target polynucleotide refers to the 10, 15, 20, 25, 30, 35, 40, 45 or 50, most terminal nucleotides on the 5′ end of the target polynucleotide. In some embodiments, a primer binding sequence is located within 10 nucleotides the 5′ end region of the target polynucleotide. In some embodiments, a primer binding sequence is located within 25 nucleotides the 5′ end region of the target polynucleotide. In some embodiments, a primer binding sequence is located within 50 nucleotides the 5′ end region of the target polynucleotide.

In some embodiments, a primer binding sequence attached to the 3′ terminal nucleotide of the target polynucleotide. In some embodiments, a primer binding sequence is located within the 3′ end of the target polynucleotide. In some embodiments, the 3′ end of the target polynucleotide refers to the 7, 8, 9, 10, 11, 12, or 13 most terminal nucleotides on the 3′ end. In some embodiments, the 3′ end of the target polynucleotide refers to the 10, 15, 20, 25, 30, 35,40, 45 or 50 most terminal nucleotides on the 3′ end. In some embodiments, the 3′ end of the target polynucleotide refers to the 10, 15, 20, 25, 30, 35, 40, 45 or 50 most terminal nucleotides on the 3′ end target polynucleotide. In some embodiments, a primer binding sequence is located within 10 nucleotides the 3′ end of the target polynucleotide. In some embodiments, a primer binding sequence is located within 25 nucleotides the 3′ end of the target polynucleotide. In some embodiments, a primer binding sequence is located within 50 nucleotides the 3′ end of the target polynucleotide.

In some embodiments, a primer binding sequence comprises a nickase recognition sequence. In some embodiments, a primer binding sequence comprises a complete or partial nickase recognition sequence (as described below). In some embodiments, a primer binding sequence comprises a reverse complement of a nickase recognition sequence. In some embodiments, the nickase recognition sequence is selected from 5′-CCTCAGC-3′ (Nb.BbvCI), 5′-GCTCTTC-3′ (Nt.BspQI), 5′-CCT-3′, 5′-CCG-3′, or 5′-CCA-3′ (Nt.CviPII), 5′-GAGTCNNNNN-3′ (Nt.BstNBI), 5′-GCAATGNN-3′ (Nb.BsrDI), 5′-GCAGTGNN-3′(Nb.BtsI), 5′-GGATCNNNNN-3′ (Nt.AlwI), 5′-GAATGCN-3′ (Nb.BsmI), 5′-CACGAG-3′ (Nb.BssSI), and 5′-GTCTCNN-3′ (Nt.BsmAI). In some embodiments, a primer binding sequence comprises ‘5-CCTCAGC-3’, which can be nicked by Nb.BbvCI. In some embodiments, a primer binding sequence comprises a sequence of 5′-CTCCTCCTCA-3′ (SEQ ID NO 1), which comprises a partial Nb.BbvCI nickase recognition sequence (bold). In some embodiments, a primer binding sequence is within 5, 10, 25, or 50 base pairs of the sequence of interest or the 5′ end of the target polynucleotide. In some embodiments, a primer binding sequence is within 5, 10, 25, or 50 base pairs of the sequence of interest or the 3′ end of the target polynucleotide.

In some embodiments, the target polynucleotide comprises a primer binding sequence and an additional sequence. In some embodiments, the additional sequence is the reverse complement of a primer binding sequence. In some embodiments, the additional sequence, when reverse transcribed, is the same sequence as the primer binding sequence. Thus, in some embodiments, the additional sequence is a primer binding sequence on the reverse complement of the target polynucleotide. In some embodiments, the additional sequence comprises the reverse complement of a complete or partial nickase recognition sequence described above. In some embodiments, the additional sequence comprises a sequence of 5′-TGAGGAGGAG-3′ (SEQ ID NO: 2). In some embodiments, the additional sequence is located within the 3′ end region of the target polynucleotide. In some embodiments, the additional sequence located within the 5′ end region of the target polynucleotide. In some embodiments, the additional sequences comprise a sequence that is not the reverse complement of the primer binding sequence. In some embodiments, the primer binding sequence and the reverse complement of the additional sequence are orthogonal sequences. The term “orthogonal” describes two or more nucleic acid molecules that lack sufficient complementarity to appreciably hybridize with one another as compared to nucleic acid molecules that comprise complementary sequences.

In some embodiments, the target polynucleotide comprises a sequence of 5′-TGAGGAGGAGNNN . . . NNNCTCCTCCTCA-3′ (SEQ ID NOs: 6-7), where NNN . . . NNN is the sequence of interest. The sequence of interest, in some embodiments, is flanked by primer binding sequences (5′-TGAGGAGGAG-3′ (SEQ ID NO: 2) and 5′-CTCCTCCTCA-3′ (SEQ ID NO: 1)) that are reverse complements of one another. The skilled person will understand that the length of the sequence of interest may vary. In some embodiments, the sequence of interest is 15-50, 15-100, 15-150, 15-200, 15-300, 20-50, 20-100, 20-150, 20-200, 20-300, 50-100, 50-150, 50-200, 50-300, 100-150, 100-200, or 100-300 nucleotides in length, In some embodiments, the sequence of interest is at least 15 nucleotides in length. In some embodiments, the sequence of interest is at least 18 nucleotides in length. In some embodiments, the sequence of interest is at least 20 nucleotides in length. In some embodiments, the sequence of interest is of sufficient length that it can be detected using Cas12 or Cas13, or a variant thereof.

### Attachment of Primer Binding Sequences to the Target Polynucleotide

In some embodiments, the primer binding sequence are attached to the target polynucleotide. In some embodiments, the primer binding sequence(s) are attached to the target polynucleotide using known methods e.g., restriction enzyme digestion/ligation or blunt end ligation. In some embodiments, the primer binding sequence(s) are attached to the target polynucleotide using isothermal ligation methods described in Gibson et al. “Nature methods 6.5 (2009): 343-345, which is incorporated by reference in its entirety. In some embodiments, the primer binding sequences are attached to the target polynucleotide using splint ligation as described in Kershaw et al. Recombinant and In Vitro RNA Synthesis. Humana Press, Totowa, NJ, 2013. 257-269. In some embodiments, the first primer binding sequence is attached to the 5′ terminal of the target polynucleotide and an additional sequence, comprising the reverse complement of a second primer binding sequence, is attached to the 3′ terminal of the target polynucleotide. In some embodiments, the first primer binding sequence is attached to the 3′ terminal of the target polynucleotide and an additional sequence, comprising the reverse complement of a second primer binding sequence, is attached to the 5′ terminal of the target polynucleotide. In some embodiments, attached of the primer binding sequences to the target polynucleotide is performed in isothermal and low temperature conditions. The skilled person will understand that the additional sequence can be attached to the target polynucleotide using the same methods, or substantially similar methods, as used to attach the primer binding sequence to the target polynucleotide.

### RNA Polymerase Binding Sequence

In some embodiments, detection of an amplified sequence of interest involves the use of an RNA polymerase, such as a T7 RNA polymerase. In such embodiments, the target polynucleotide further comprises an RNA polymerase binding sequence. In some embodiments, the target polynucleotide is modified to comprise an RNA polymerase binding sequence. In some embodiments, the RNA polymerase binding sequence is added to the target polynucleotide using a primer during amplification that comprises a stabilization sequence comprising an RNA polymerase binding sequence. In some embodiments, the RNA polymerase binding sequence is added to the target polynucleotide as part of the primer binding sequence or the additional sequence.

In some embodiments, the RNA polymerase binding sequence is cognate to an RNA polymerase included in the composition. In some embodiments, the RNA polymerase binding sequence is a bacterial RNA polymerase binding sequence. In some embodiments, the RNA polymerase binding sequence is a eukaryotic RNA polymerase binding sequence. In some embodiments, the RNA polymerase binding sequence is a bacteriophage RNA polymerase binding sequence. In some embodiments, the RNA polymerase binding sequence is an RNA polymerase I, RNA polymerase II, RNA polymerase III, RNA polymerase IV, RNA polymerase V, Nr virion RNA polymerase, or T7 RNA polymerase binding sequence. In some embodiments, the RNA polymerase binding sequence is a T7 RNA polymerase binding sequence.

### Primers

The methods provided herein use one or more primers to amplify the target polynucleotide. For example, the composition may comprise a primer. It should be understood that embodiments describing a “primer” encompass a “first primer,” a “second primer,” and any additional primers that may be contemplated.

### Hybridization Sequence

A primer comprises a hybridization sequence that is complementary to a primer binding sequence in a target polynucleotide. Thus, a single stranded target polynucleotide comprises (or is modified to comprise), in some embodiments, a primer binding sequence to which a hybridization sequence binds. Following binding of the primer to the single stranded target polynucleotide and subsequent polymerase-based elongation, a reverse complement of the target polynucleotide is produced. In some embodiments, the reverse complement of the target polynucleotide comprises the same primer binding sequence as the target polynucleotide. Following from this, the same primer may bind to the single stranded target polynucleotide and the reverse complement of the single stranded target polynucleotide. Elongation of the single stranded target polynucleotide and the reverse complement results in exponential amplification of the target polynucleotide.

In some embodiments, the composition comprises a primer. In some embodiments, a primer comprises a hybridization sequence. A hybridization sequence refers to a sequence that is complementary to a primer binding sequence of a target polynucleotide. In some embodiments, a hybridization sequence is at least 85% (e.g., at least 85%, at least 90%, at least 95%, or at least 99%) complementary to a primer binding sequence of a target polynucleotide. In some embodiments, a hybridization sequence is 100% complementary to a primer binding sequence of a target polynucleotide. In some embodiments, a primer comprises a hybridization sequence that is complementary to the reverse complement of an additional sequence (e.g., a primer binding sequence) of the target polynucleotide. In some embodiments, the hybridization sequence comprises a sequence that is a nickase recognition sequence. In some embodiments, the hybridization sequence comprises a sequence that is selected from 5′-CCTCAGC-3′ (Nb.BbvCI); 5′-GCTCTTC-3′ (Nt.BspQI); 5′-CCT-3′, 5′-CCG-3′, or 5′-CCA-3′ (Nt.CviPII); 5′-GAGTCNNNNN-3′ (Nt.BstNBI); 5′-GCAATGNN-3′ (Nb.BsrDI); 5′-GCAGTGNN-3′ (Nb.BtsI); 5′-GGATCNNNNN-3′ (Nt.AlwI); 5′-GAATGCN-3′ (Nb.BsmI); 5′-CACGAG-3′(Nb.BssSI); and 5′-GTCTCNN-3′ (Nt.BsmAI), or is a reverse complement of one of these nickase recognition sequences. In some embodiments, the hybridization sequence comprises a sequence that is a reverse complement of a Nb.BbvCI (5′-CCTCAGC-3′) sequence. In some embodiments, the hybridization sequence comprises a complete or partial nickase recognition sequence (or a reverse complement thereof). In some embodiments, the hybridization sequence comprises a complete or partial nickase recognition sequence (or a reverse complement thereof) and 2, 3, 4, 5, 6, 7, or 8 additional nucleotides. In some embodiments, the hybridization sequence comprises a complete or partial nickase recognition sequence (or a reverse complement therefore) and 2-3, 2-4, 2-5, 2-6, 2-7, 2-8, 2-9, 3-4, 3-5, 3-6, 3-7, 3-8, 3-9, 4-5, 4-6, 4-7, 4-8, 4-9, 5-6, 5-7, 5-8, 5-9, 6-7, 6-8, 6-9, 7-8, 7-9, or 8-9 additional nucleotides. In some embodiments, the hybridization sequence comprises a complete or partial nickase recognition sequence (or a reverse complement therefore) and 4-6 additional nucleotides. In some embodiments, the hybridization sequence comprises 8-9, 8-10, 8-11, 8-12, 9-10, 9-11, 9-12, 10-11, 10-12, or 11-12 nucleotides. In some embodiments, hybridization sequence comprises 9-11 nucleotides. In some embodiments, hybridization sequence comprises 7, 8, 9, 10, 11, 12, 13, 14, 15, 16, 17, 18, 19, 20, 21, 22, 23, 24, 25, 26, 27, 28, 29, or 30 nucleotides. In some embodiments, the hybridization sequence comprises 8-13, 8-14, 8-15, 8-16, 8-17, 8-18, 8-19, 8-20, 8-21, 8-22, 8-23, 8-24, 8-25, 8-26, 8-27, 8-28, 8-29, or 8-30 nucleotides. In some embodiments, the hybridization sequence comprises 3, 4, 5, 6, 7, 8, 9, 10, 11, 12, 13, or 14 nucleotides. In some embodiments, the hybridization sequence comprises 9, 10, or 11 nucleotides. In some embodiments, the hybridization sequence comprises 9-10, 9-11, or 10-11 nucleotides. In some embodiments, the hybridization sequence comprises 10 nucleotides.

In some embodiments, a hybridization sequence comprises a full nickase recognition sequence. In some embodiments, a primer binding sequence comprises a full nickase recognition sequence. In some embodiments, the primer hybridization sequence comprises a partial nickase recognition sequence and a primer binding sequence comprises a full nickase recognition sequence. In some embodiments, a primer comprises a first partial nickase recognition sequence and the primer binding sequence comprises a second partial recognition sequence, wherein the first partial nickase recognition sequence and the second partial nickase recognition sequence together form a complete nickase recognition sequence that is produced during elongation.

In some embodiments, the primer binding sequence of the target polynucleotide and the additional sequence are not reverse compliments of on another. In some embodiments, the primer binding sequence of the target polynucleotide and the additional sequence are orthogonal to one another. In some embodiments, the composition comprises a second primer with a hybridization sequence that is complementary to the reverse complement of the additional sequence of the target polynucleotide and is not complementary to the primer binding sequence of the target polynucleotide.

### Stabilization Sequence

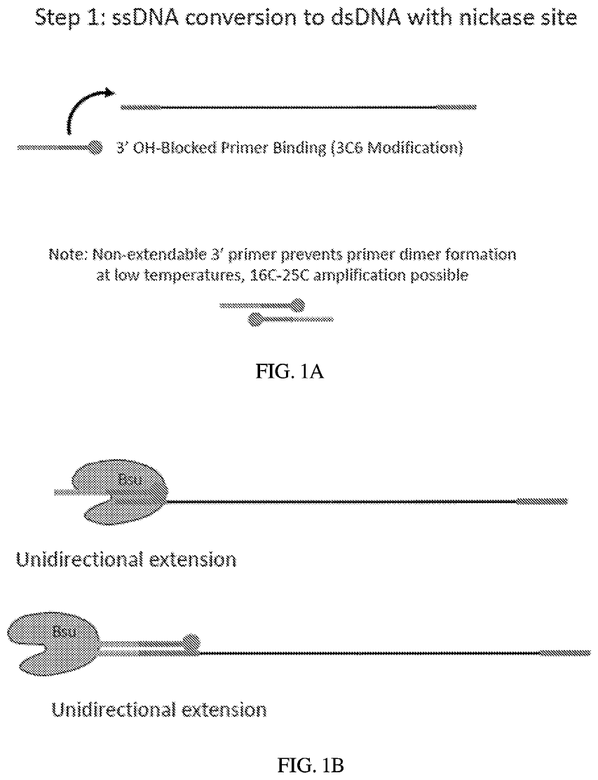

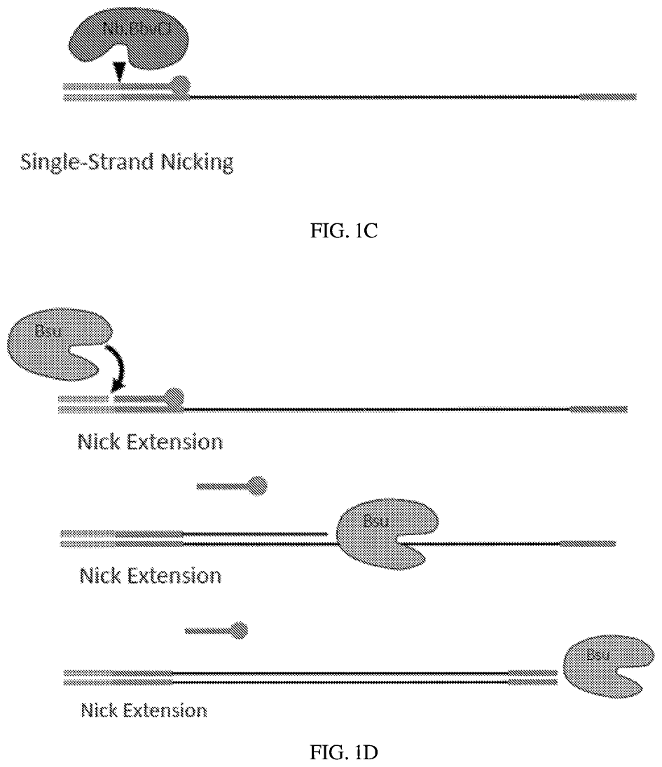

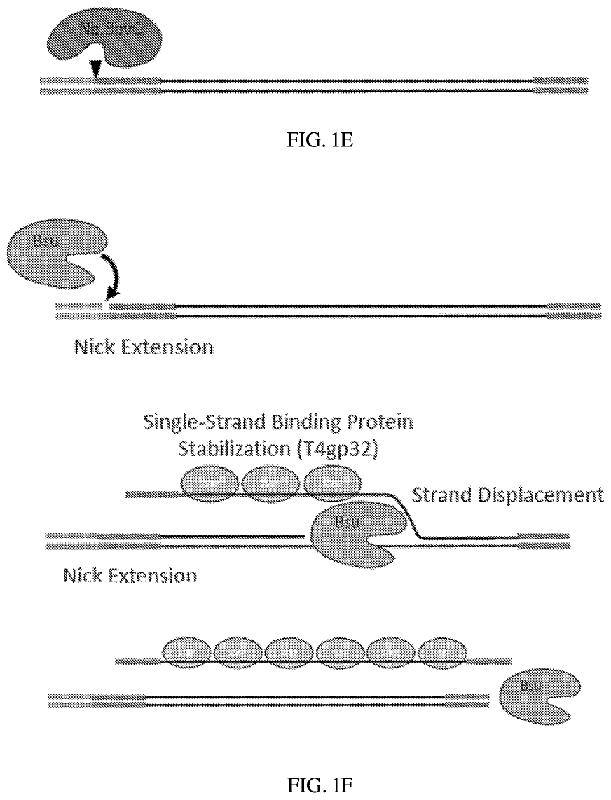

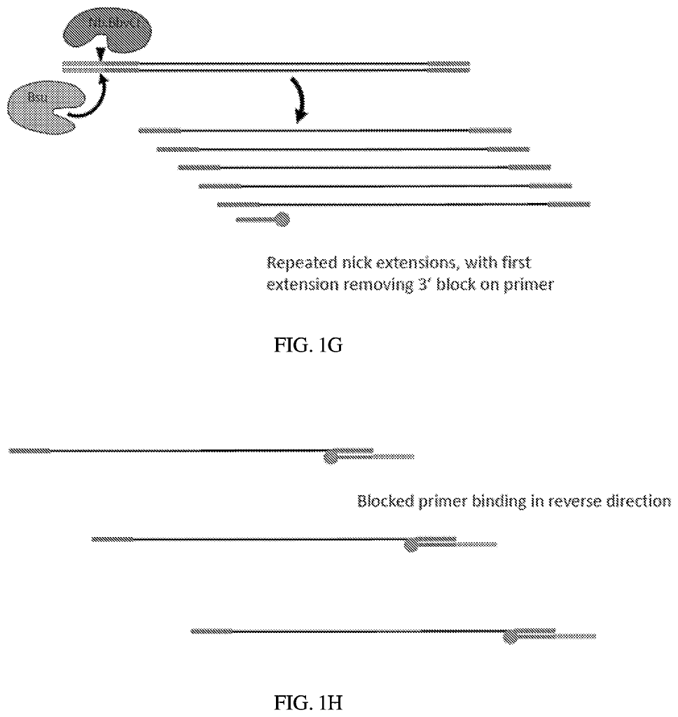

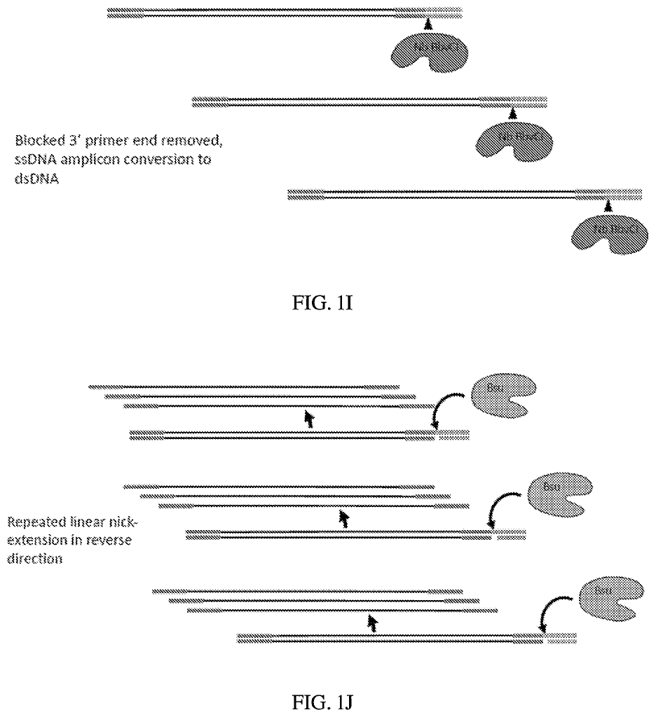

In some embodiments, a primer further comprises a stabilization sequence that extends from the 5′ end of the primer. The stabilization sequence provides a template for elongation of the target polynucleotide (or reverse complement thereof) following primer binding (FIG. 1B). Additionally, in some embodiments, the stabilization sequence remains in a duplex with the elongated target polynucleotide after nickase cleavage such that the primer can be elongated by a DNA polymerase (DNAP) (see FIG. 1C and FIG. 1D). In some embodiments, the stabilization sequence comprises 6-30 nucleotides. In some embodiments, the stabilization sequence is 6, 7, 8, 9, 10, 11, 12, 13, 14, 15, 16, 17, 18, 19, 20, 21, 22, 23, 24, 25, 26, 27, 28, 29, or 30 nucleotides. In some embodiments, the stabilization sequence is 16-22 nucleotides. In some embodiments, the stabilization sequence is 18 nucleotides. In some embodiments, the stabilization sequence further comprises an RNA polymerase binding sequence (e.g., a T7 Polymerase binding sequence). In some embodiments, the stabilization sequence comprises a sequence of 5′-GAAGGTCGAAGATCGC-3′ (SEQ ID NO: 3).

In some embodiments, a primer comprises a sequence of 5′-GAAGGTCGAAGATCGCTGAGGAGGAG-3′ (bold is hybridization sequence) (SEQ ID NO: 4).

### Blocking Molecule

In some embodiments, a primer, a first and/or second primer comprises a blocking molecule on the last nucleotide of the 3′ end of the primer. In some embodiments, the blocking molecule blocks elongation of the primer (e.g., stops elongation from proceeding) in the 3′ direction by chemically modifying the 3′ OH group of the 3′ terminal nucleotide of the primer. In some embodiments, the 3′ OH chemical modification blocks the strand displacing polymerase from adding an additional nucleotide to the 3′ terminal nucleotide of the primer. In some embodiments, the 3′ blocking molecule may also inhibit exponential amplification of primer dimers.

In some embodiments, the 3′ blocking molecule, when bound to the 3′ terminal of a primer, blocks elongation of the primer in the 3′ direction. In some embodiments, the blocking molecule binds to the 3′ OH group of the 3′ terminal nucleotide of the primer. In some embodiments, the blocking molecule is selected from the group consisting of:

(1) 3′ddNTP (dideoxynucleotide triphosphates (e.g., ddTTP, ddATP, ddGTP, or ddCTP) that do not have the 3′ OH group required for elongation),

(2) 3′ Inverted dT (has a 3′-3′ linkage that inhibits elongation),

(3) a 3′ carbon chain spacer (a carbon chain bound to the 3′ OH group required for elongation. The spacer may be 4C. 5C, 6C, 7C, 8C, 9C, 10C, 11C, 12C or more),

(4) 3′ hexanediol (a C6 glycol chain binds to the 3′ OH group required for elongation),

(5) 3′ amino (is a 4C, 5C, 6C, 7C, 8C, 9C, 10C, 11C, 12C or more carbon chain spacer with a methoxy group on C1, and an NH2 group bound to the last carbon of the spacer (4C, 5C, 6C. 7C, 8C, 9C, 10C, 11C, 12C or more). The amino spacer binds to the 3′OH group required for elongation), and

(6) 3′ phosphorylation (a phosphate group bound to the 3′ OH required for elongation, so the 5′ phosphate group from the proceeding nucleotide cannot bind to the 3′ OH of the 3′ terminal nucleotide on the primer).

In some embodiments, the blocking molecule is 3′ hexanediol.

### Nickase

The methods provided herein use nickases to (1) nick double stranded DNA comprising the primer to allow removal of the 3′ blocking group during elongation of the primer, and (2) nick double stranded polynucleotide to allow for strand displacement during elongation. Nick, nicking, and nicked all refer to cleaving one strand of a double stranded DNA molecule (e.g., a target polynucleotide) by a nickase. The nickases used herein may nick specific nickase recognition sequences. The term, cognate nickase is used to describe the pairing nickase with its corresponding nickase recognition sequence. For example, Nb.BbvCI is cognate to 5′-CCTCAGC-3′.

In some embodiments, the composition further comprises a nickase. A nickase is an enzyme that is capable of binding to a double stranded polynucleotide and nicking (i.e., cleaving) just one strand. Nickases are well known in the art and described in Zhang et al. Protein expression and purification 69.2 (2010): 226-234; Higgins et al. Nucleic acids research 29.12 (2001): 2492-2501; Morgan et al. (2000): 1123-1125; Xu et al. Proceedings of the National Academy of Sciences 98.23 (2001): 12990-12995; Heiter et al. Journal of molecular biology 348.3 (2005): 631-640; Zhu et al. Journal of molecular biology 337.3 (2004): 573-583; and Samuelson et al. Nucleic acids research 32.12 (2004): 3661-3671, each of which is incorporated by reference in its entirety. In some embodiments, the nickase is cognate to the nickase recognition sequence on target polynucleotide and/or primer. In some embodiments, the nickase nicks the double stranded target polynucleotide product, which contributes to low (e.g., 4-30° C. or 12-30° C.) temperature, isothermal displacement of the strand by the polymerase. In some embodiments, the nickase is selected from the group consisting of Nt. BspQI, Nt.CviPII. Nt.BstNBI, Nb.BsrDI, Nb.BtsI, Nt.AlwI, Nb.BbvCI, Nt.BbvCI, Nb.BsmI, Nb.BssSI, and Nt.BsmAI. In some embodiments, the nickase is Nb.BbvCI. Nickases are may be associated with recognition sequences: 5′-CCTCAGC-3′ (Nb.BbvCI), 5′-GCTCTTC-3′ (Nt.BspQI), 5′-CCT-3′, 5′-CCG-3′, or 5′-CCA-3′ (Nt.CviPII), 5′-GAGTCNNNNN-3′ (Nt.BstNBI), 5′-GCAATGNN-3′ (Nb.BsrDI), 5′-GCAGTGNN-3′ (Nb.BtsI), 5′-GGATCNNNNN-3′ (Nt.AlwI), 5′-GAATGCN-3′ (Nb.BsmI), 5′-CACGAG-3′ (Nb.BssSI), and 5′-GTCTCNN-3′ (Nt.BsmAI).

In some embodiments, the nickase recognition sequence is a complete nickase recognition sequence. A complete nickase recognition sequence is a sequence that would be recognized and nicked by a nickase. A complete nickase recognition sequence depends upon the cognate nickase. For example, if Nb.BbvCI is the nickase then 5′-CCTCAGC-3′ would be a complete nickase recognition sequence. If Nt.CviPII is the nickase then 5′-CCT-3′, 5′-CCG-3′, or 5′-CCA-3′ would be the recognition sequence. In some embodiments, the nickase recognition sequence is a partial nickase recognition sequence. A partial recognition sequence is a sequence that has a portion of a complete recognition sequence. A partial nickase recognition sequence depends on the nickase. For example, if Nb.BbvCI is the nickase then 5′-CCT-3′ would be a partial nickase recognition sequence. However, if Nt.CviPII is the nickase then 5′-CCT-3′ would not be a partial recognition sequence. A nickase partial recognition sequence contains contiguous nucleotides from the complete nickase recognition sequence, wherein the partial recognition sequence comprises the first or last nucleotide in the complete recognition sequence. In some embodiments, a partial nickase recognition sequence does not comprise 1, 2, 3, 4, 5, 6, 7, 8, or 9 consecutive nucleotides that are in the complete nickase recognition sequence. In some embodiments, the partial nickase recognition sequence does not comprise at least 1 (e.g., at least 1, at least 2, at least 3, at least 4, at least 5, at least 6, at least 7, at least 8, or at least 9) nucleotides that are in the complete recognition sequence. In some embodiments, the partial nickase recognition sequence does not comprise 1-2, 1-3, 1-4, 1-5, 1-6, 1-7, 1-8, 1-9, 2-3, 2-4, 2-5, 2-6, 2-7, 2-8, 2-9, 3-4, 3-5, 3-6, 3-7, 3-8, 3-9, 4-5, 4-6, 4-7, 4-8, 4-9, 5-6, 5-7, 5-8, 5-9, 6-7, 6-8, 6-9, 7-8, 7-9, or 8-9 of the nucleotides that are in the complete nickase recognition sequence.

In some embodiments, a hybridization sequence comprises a full nickase recognition sequence. In some embodiments, a primer binding sequence comprises a full nickase recognition sequence. In some embodiments, the primer hybridization sequence comprises a partial nickase recognition sequence and a primer binding sequence comprises a full nickase recognition sequence. In some embodiments, a primer comprises a first partial nickase recognition sequence and the primer binding sequence comprises a second partial recognition sequence, wherein the first partial nickase recognition sequence and the second partial nickase recognition sequence together form a complete nickase recognition sequence that is produced during elongation. For example, in some embodiments, a primer binding sequence comprises a sequence of 5′-CTCCTCCTCA-3′ (SEQ ID NO 1), which comprises a partial Nb.BbvCI nickase recognition sequence (bold); in these embodiments, a primer may comprise a hybridization region that is the reverse complement of 5′-CTCCTCCTCA-3′ (SEQ ID NO: 1) and the primer further comprises the reverse complement of remainder of the nickase recognition sequence (bold) (5′-

CGTGAGGAGGAG-3′ (SEQ ID NO: 5)). Thus, elongation of the target polynucleotide, in some embodiments, results in a complete nickase recognition sequence.

### Strand Displacing Polymerase

The methods provided herein use strand displacing polymerases to amplify the target polynucleotide. Thus, in some embodiments, the composition further comprises a strand displacing polymerase. A strand displacing polymerase is able to displace downstream DNA during elongation. In some embodiments, the strand displacing polymerase has elongation activity at low temperature (e.g., 4-25° C.). In some embodiments, the strand displacing polymerase has elongation activity at 13-25° C. 13-24° C. 13-23° C. 13-22° C., 13-21° C., 13-20° C. 13-19° C., 13-18° C., 13-17° C., 13-16° C., or 13-15° C. In some embodiments, the strand displacing enzyme has elongation activity at 4° C. 5° C., 6° C. 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C. 27° C. 28° C. 29° C., or 30° C. In some embodiments, the strand displacing polymerase has elongation activity at room temperature. In some embodiments, the strand displacing polymerase is selected from the group consisting of Bsu DNA Polymerase I (Bsu), phi29, Bst DNA Polymerase, Klenow Large Fragment, Klenow Exo-, Bsu Large Fragment, Isopol, and Isopol SD+, or variants thereof. In some embodiments, the strand displacing polymerase is Bsu or a variant thereof. In some embodiments, the strand displacing polymerase is Klenow or a variant thereof.

### Single Stranded Binding Protein

The methods provided herein use the single strand binding proteins to stabilize the displaced strand during elongation. Single stranded binding proteins, and application thereof, are further described in Kur et al. Acta Biochimica Polonica 52.3 (2005): 569-574, which is incorporated by reference in its entirety. In some embodiments, the composition further comprises a single stranded binding protein (SSBP). In some embodiments, the SSBP binds to the DNA strand that is displaced by the strand displacing polymerase. In some embodiments, the SSBP is selected from the group consisting of T4 Gene 32 Protein (T4gp32), EcoSSB, TaqSSB, and TthSSB as described in Kur et al. Acta Biochimica Polonica 52.3 (2005): 569-574 and Alberts et al. Nature 227. 1313-1318 (1970), which are both incorporated by reference in its entirety. In some embodiments, the SSBP is T4gp32.

### Buffer

The methods and compositions provided herein comprise buffers that provide reaction conditions suitable for amplification and/or detection of a target polynucleotide. In some embodiments, the buffer comprises components that a skilled person would understand to be in a buffer for DNA amplification. In some embodiments, the composition further comprises a buffer in which isothermal, low temperature amplification of the target polynucleotide can occur. In some embodiments, the buffer comprises deoxynucleotides (dNTPs). In some embodiments, the buffer comprises Tris or acetate. In some embodiments, the buffer comprises potassium ions (K⁺). In some embodiments, the buffer comprises potassium acetate or potassium chloride. In some embodiments, the buffer comprises magnesium ions (Mg²⁺). In some embodiments, the buffer comprises magnesium chloride. In some embodiments, the buffer comprises a polymerase chain reaction enhancer (e.g., Dimethyl sulfoxide (DMSO), Glycerol, Formamide, Bovine Serum Albumin, Ammonium sulfate, polyethylene glycol, gelatin, tween 20, triton X-100, or N.N,N-trimethylglycine (betaine)). In some embodiments, T7 RNA polymerase is active in the buffer. In some embodiments, Cas13 polymerase is active in the buffer. In some embodiments, Cas12 is active in the buffer. In some embodiments, SHERLOCK is active in the buffer. In some embodiments, the buffer comprises CutSmart Buffer.

### Detection of the Amplified Polynucleotide

In some aspects, the present disclosure provides method for producing multiple copies of a target polynucleotide by amplification then detecting the multiple copies of the target polynucleotide.

As discussed below, detection may occur in a one pot reaction or a two pot reaction. In a one pot reaction, in some embodiments, the reagents for target polynucleotide amplification and detection are present throughout incubation. In one pot reactions, in some embodiments, the copies of the target polynucleotide are transcribed to RNA then detected by an RNA binding protein (e.g., Cas13). Cas13 cleaves the RNA during detection a collateral Cas13 activity also cleaves the detector polynucleotide. Collateral Cas protein activity is non-specific cleavage of nucleic acids (e.g., a detector polynucleotide) that occurs when the crRNA: Cas complex is cleaving a target specified by the crRNA (e.g., the target polynucleotide). Collateral activity is discussed in detail in Varble et al. “Trends in Genetics 35.6 (2019): 446-456, which is incorporated by reference in its entirety. In a two pot reaction, in some embodiments, the reagents for target nucleotide amplification are added to the composition first, followed by an incubation. After the incubation (and presumably amplification of the target polynucleotide), the reagents for detection are added to the composition. In some embodiments, the reagents for detection comprise DNA binding proteins (e.g., Cas12). The Cas12 cleaves the copies of the target polynucleotide during detection. Hence, the Cas12 may added after incubation and amplification as to not disrupt the amplification process.

### One Pot Reaction

In some embodiments, both amplification of the target polynucleotide and detection of copies of the target polynucleotide are be performed in the same reaction (e.g., one pot reaction). The copies of the target polynucleotide, in some embodiments, are transcribed into RNA then detected by an RNA binding molecule (e.g., Cas13). In some embodiments, the RNA binding molecule specifically recognizes RNA transcribed from copies of the target polynucleotide or

RNA transcribed from the reverse complement of the target polynucleotide. In such embodiments, the composition further comprises an RNA polymerase (as described above), and the target polynucleotide is modified to comprise an RNA polymerase binding sequence as described above. In some embodiments, the RNA binding molecule is a protein that specifically recognizes RNA transcribed from the copies of the target polynucleotide. In some embodiments, the RNA binding molecule is a nuclease. In some embodiments, the nuclease, when bound to the RNA, has collateral nuclease activity that activates the detectable molecule (described below). In some embodiments, the nuclease is a CRISPR Cas nuclease that binds to RNA. In some embodiments, the nuclease is Cas13. In some embodiments, the Cas13 is selected from the group consisting of Cas13a, Cas13b, Cas13c, or Cas13d. In some embodiments, the Cas13 is LwaCas13a, LbaC13a, CcaCas13b, PsmCas13b, or AsCas12a as described in Kellner et al. Nature protocols 14.10 (2019): 2986-3012, which is incorporated by reference in its entirety. In some embodiments, the Cas13 is LbuCas13a. In some embodiments, the composition further comprises a crRNA that is complementary to the RNA transcribed from the multiple copies of the target polynucleotide. In some embodiments, the crRNA forms a complex with the CRISPR Cas (e.g., Cas13a), and directs the CRISPR Cas to the RNA for degradation.

In some embodiments, the composition comprises a detector polynucleotide. The detector polynucleotide is used to detect copies of the target polynucleotide and/or the reverse complement of the target polynucleotide. In some embodiments, the detector molecule comprises a detector polynucleotide that comprises a quencher molecule on one end (5′ or 3′) and a detectable molecule (e.g., Iowa Black-RQ, Black Hole Quencher-1, Black Hole Quencher-2, and Iowa Black-ZEN FQ) a fluorophore (e.g., FAM, 6-FAM, Tye 563, Cy 3, ATTO 550, TAMRA 583, ATTO 565, ROX, ATTO Rho 101, TEX 615, Texas Red-X, TYE 665 or Cy 5) on the other end. In some embodiments, the detector polynucleotide detects binding and cleavage of the multiple copies of the target polynucleotide by Cas13. A detector polynucleotide is cleaved by the collateral activity of the CRISPR Cas guide RNA complex (i.e., crRNA: Cas12) when the complex binds and cuts copies of the target polynucleotide, as described in Kellner et al. Nature protocols 14.10 (2019): 2986-3012. In some embodiments, the quencher molecule is selected from the group consisting of Iowa Black-RQ, Black Hole Quencher-1, Black Hole Quencher-2, and Iowa Black-ZEN FQ. In some embodiments, the fluorophore is selected from the group consisting of Tye 563, Cy 3, ATTO 550, TAMRA 583, ATTO 565, ROX, ATTO Rho 101, TEX 615, Texas Red-X, TYE 665 and Cy 5, and the quencher molecule is selected from the group consisting of Iowa Black and Black Hole quencher-2. In some embodiments, the fluorophore is selected from the group consisting of FAM. 6-FAM, ATTO 488, TET i539, JOE, HEX, and ATTO 532 and the quencher molecule is selected from the group consisting of Black Hole Quencher-1, and lowa Black-ZEN FQ. In some embodiments, the detector polynucleotide comprises a 6-FAM fluorophore and Black Hole Quencher-1. In some embodiments, a detector polynucleotide comprises 5, 6, 7, 8, 9, 10, 11, 12, 13, 14, 15, 16, 17, 18, 19, 20 or more nucleotides. In some embodiments, a detector polynucleotide comprises 5-10, 5-15, 5-20, 10-15, 10-20, or 15-20 nucleotides. In some embodiments, a detector polynucleotide comprises a polyU (poly-Uracil) sequence (e.g., 5-20 contiguous uracil nucleotides). In some embodiments, the detector polynucleotide comprises 5, 6, 7, 8, 9, 10, 11, 12, 13, 14, 15, 16, 17, 18, 19, 20 or more contiguous uracil nucleotides. In some embodiments, the detector polynucleotide comprises 5-10, 5-15, 5-20, 10-15, 10-20, or 15-20 contiguous uracil nucleotides. In some embodiments, the detector polynucleotide can be cleaved by Cas13 collateral activity.

As described above, in one pot detection embodiments, the target polynucleotide may comprise or be modified to comprise an RNA polymerase binding sequence, which is used to transcribe the amplified copies of the target polynucleotide into RNA for detection by an RNA binding protein (e.g., Cas13). To perform transcription, in some embodiments, the one pot reaction further comprises an RNA polymerase. In some embodiments, the RNA polymerase is a cognate RNA polymerase. A cognate RNA polymerase binds to an RNA polymerase binding sequence on the target polynucleotide. In some embodiments, the RNA polymerase is a bacterial RNA polymerase. In some embodiments, the RNA polymerase is a eukaryotic RNA polymerase. In some embodiments, the RNA polymerase is a bacteriophage RNA polymerase. In some embodiments, the RNA polymerase is RNA polymerase I, RNA polymerase II, RNA polymerase III, RNA polymerase IV, RNA polymerase V. Nr virion RNA polymerase, or T7 RNA polymerase. In some embodiments, the RNA polymerase is a T7 RNA polymerase.

### Two Pot Reaction

In some embodiments, amplification of the target polynucleotide and detection of copies of the target polynucleotide are performed in separate or consecutive reactions. In such reactions, copies of the target polynucleotide may be directly detected using a DNA binding molecule. In some embodiments, the composition comprises a DNA binding molecule that specifically recognizes the copies of the target polynucleotide or the reverse complement of the target polynucleotide. In some embodiments, the DNA binding molecule is a nuclease. In some embodiments, the nuclease, when bound to DNA, has collateral nuclease activity that cleaves a detector molecule (described below). In some embodiments, the nuclease is a CRISPR Cas nuclease that binds to DNA. In some embodiments, the nuclease is Cas 12. In some embodiments, the Cas 12 is selected from the group consisting of Cas12a or Cas12b. In some embodiments, the Cas 12 is LbaCas 12a or AsCas 12a. In some embodiments, the composition further comprises a guide RNA that is complementary to the target polynucleotide or amplicon thereof. The guide RNA forms a complex with the CRISPR Cas (e.g., Cas12a), and directed the CRISPR Cas to the target polynucleotide or amplicon thereof for degradation. In some embodiments, the detection in the two-pot reaction may be achieved using Cas13 as described above except that the detection reagents are added to the composition after incubation.

In some embodiments, the composition comprises a detector polynucleotide that comprises a quencher the 5′ or 3′ terminal and a fluorophore on the other terminal. A detector polynucleotide is cleaved by the CRISPR Cas guide RNA complex upon binding and cutting of the target polynucleotide or amplicons thereof. In some embodiments, the detector polynucleotide quencher is selected from the group consisting of Iowa Black-RQ, Black Hole Quencher-1, Black Hole Quencher-2, and Iowa Black-ZEN FQ. In some embodiments, the fluorophore is selected from the group consisting of Tye 563, Cy 3, ATTO 550, TAMRA 583, ATTO 565, ROX, ATTO Rho 101, TEX 615, Texas Red-X, TYE 665 and Cy 5, and the quencher is selected from the group consisting of Iowa Black and Black Hole quencher-2. In some embodiments, the fluorophore is selected from the group consisting of FAM, 6-FAM, ATTO 488, TET i539, JOE, HEX, and ATTO 532 and the quencher is selected from the group consisting of Black Hole Quencher-1, and lowa Black-ZEN FQ. In some embodiments, the detector polynucleotide comprises a 6-FAM fluorophore and Black Hole Quencher-1. In some embodiments, the detector polynucleotide is added to the composition at the same time the CRISPR Cas nuclease is added to the composition. In some embodiments, a detector polynucleotide comprises 5, 6, 7, 8, 9, 10, 11, 12, 13, 14, 15, 16, 17, 18, 19, 20 or more nucleotides. In some embodiments, a detector polynucleotide comprises 5-10, 5-15, 5-20, 10-15, 10-20, or 15-20 nucleotides. In some embodiments, the detector polynucleotide can be cleaved by Cas12 collateral activity.

In some embodiments, detection is accomplished using SHERLOCK, as described below

### Sherlock Detection

In some embodiments, the multiple copies of the target polynucleotide are detected using the specific high-sensitivity enzymatic reporter unlocking (SHERLOCK) system as described in Kellner et al. Nature protocols 14.10 (2019): 2986-3012, which is incorporate by reference in its entirety. A SHERLOCK system may comprise an RNA guide for a Cas12a or Cas12b enzyme targeting a sequence within the multiple copies of the target polynucleotide, such that the presence of the amplified target polynucleotide DNA induces collateral nuclease activity of the Cas12a or Cas12b enzyme. A SHERLOCK system may also comprise an RNA guide for a Cas13a or Cas13b enzyme targeting a sequence within the multiple copies of the target polynucleotide, such that the presence of RNA transcribed by an RNA polymerase using the amplified target polynucleotide as a template induces collateral nuclease activity of the Cas13a or Cas13b enzyme. For both systems, detection of the nuclease activity of Cas12a, Cas12b, Cas13a, or Cas 13b may be achieved by the cleavage of a target polynucleotide comprising a sequence flanked by a detectable molecule and a quencher molecule. In some embodiments, a Cas13 based SHERLOCK assay may be used in the one pot reaction. In some embodiments, a Cas12 or Cas13 based SHERLOCK assay may be used in the two-pot reaction.

### Methods

In some aspects, the present disclosure provides methods of producing multiple copies of a target polynucleotide using the compositions described herein. Multiple copies, as used herein, refers to at least 1 copy (e.g., at least 2, at least 10, at least 100, at least 10³, at least 10⁴, at least 10⁵, at least 10⁶, at least 10⁷, at least 10⁸, or at least 10⁹ copies) of the target polynucleotide. In some embodiments, the multiple copies of the target polynucleotide are sufficient to be detected using a DNA or an RNA binding protein (e.g., SHERLOCK). In some embodiments, the method of producing multiple copies of the target polynucleotide is exponential amplification.

In some embodiments, the method involves incubating the composition as described herein in a buffer to produce multiple copies of the target polynucleotide. In some embodiments, the buffer is sufficient to support amplification of the target polynucleotide to make multiple copies of the target polynucleotide. In some embodiments, amplification of the target polynucleotide can begin when at least the following components are combined into the buffer: the target polynucleotide, the primer, the SSBP, the nickase, and the strand displacing polymerase.

In some embodiments, incubation is in isothermal conditions (i.e., isothermal incubation). Isothermal conditions occur at a temperature that does not change, or changes minimally, throughout the incubation. In some embodiments, a room temperature incubation is an isothermal condition. In some embodiments, an isothermal conditions can vary in temperature by about 2, 3, 4, 5, or 6 degrees Celsius. In some embodiments, fluctuations in temperature during an isothermal incubation are not required for amplification or detection of the target polynucleotide. In some embodiments, incubation is at about 1° C. to about 99° C. In some embodiments, incubation is at 4° C. to 50° C. In some embodiments, incubation is at 4° C. to 37° C. In some embodiments, incubation is at 4° C. to 30° C. In some embodiments, incubation is at 4° C. to 25° C. In some embodiments, incubation is at 4° C. to 20° C. In some embodiments, incubation is at 4° C. to 15° C. In some embodiments, incubation is at 4° C. to 10° C. In some embodiments, incubation is at 12° C. to 30° C. In some embodiments, incubation is at 16° C. to 30° C. In some embodiments, incubation is at 12° C. to 25° C. In some embodiments, incubation is at 13° C. to 25° C. In some embodiments, incubation is at 14° C. to 25° C. In some embodiments, incubation is at 15° C. to 25° C. In some embodiments, incubation is at 16° C. to 25° C. In some embodiments, incubation is at 16° C. to 22° C. In some embodiments, the incubation is at about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30° C.

In some embodiments, incubation is at least 10 minutes (e.g., at least 10 minutes, at least 20 minutes, at least 30 minutes, at least 45 minutes, at least 1 hour, at least 1.5 hours, at least 2 hours, at least 3 hours, at least 4 hours, at least 6 hours, or at least 12 hours). In some embodiments, incubation is 10-30 minutes, 10-50 minutes, 10-60 minutes, 30-60 minutes, 30-90 minutes, 30-120 minutes, 30-180 minutes, 30-240 minutes. In some embodiments, incubation is 1-2 hours, 1-3 hours, 1-4 hours, 1-6 hours, 2-3 hours, 2-4 hours, 2-5 hours, 2-6 hours, 3-4 hours, 3-5 hours, 3-6 hours, 4-5 hours, or 4-6 hours. In some embodiments, the incubation is about 2 hours.

In some embodiments, the method further comprises detecting the multiple copies of the target polynucleotide. In some embodiments, the detection reagents are a Cas12 or Cas13 protein, a crRNA that is complementary to the target polynucleotide, and detector polynucleotide. In some embodiments, the detector polynucleotide is cleaved when the Cas12 or Cas13 protein cleaves the multiple copies of the target polynucleotide or RNA encoding the multiple copies of the target polynucleotide. In some embodiments, the reagents for detecting the multiple copies of the target polynucleotide are present in the composition when incubation begins (e.g., one pot reaction described above). In some embodiments, the reagents for detecting the multiple copies of the target polynucleotide are present in the composition when the sample is added (e.g., one pot reaction described above).

In some embodiments, the reagents for detecting the multiple copies of the target polynucleotide are added after a specified time of incubation (e.g., two pot reaction as described above). The specified time for adding the detection agents may be any of the incubation times described above. In some embodiments, the detection reagents are added at least 10 minutes, at least 20 minutes, at least 30 minutes, at least 40 minutes, at least 50 minutes, at least 60 minutes, at least 90 minutes, or at least 120 minutes after amplification beings. In some embodiments, the detection agent is added after about two hours of incubation.

In some embodiments, detection is performed using SHERLOCK.

In some embodiments, detection of the multiple copies of the target polynucleotide is determined visually by identifying a change in color of the composition. In some embodiments, detection of the multiple copies of the target polynucleotide is determined using a fluorometer.

### EXAMPLES

### Example 1: The CRISDA Platform is Not Robust at Lower Temperature Amplification

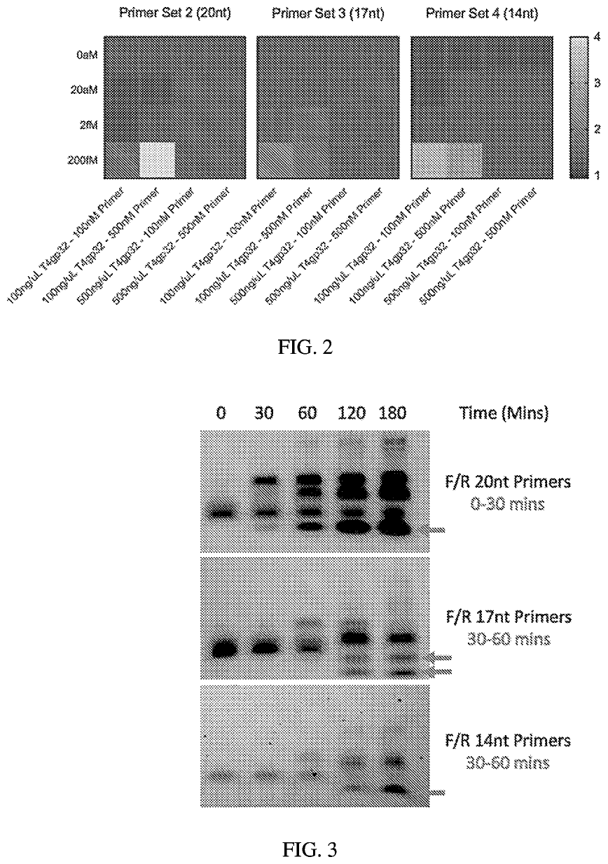

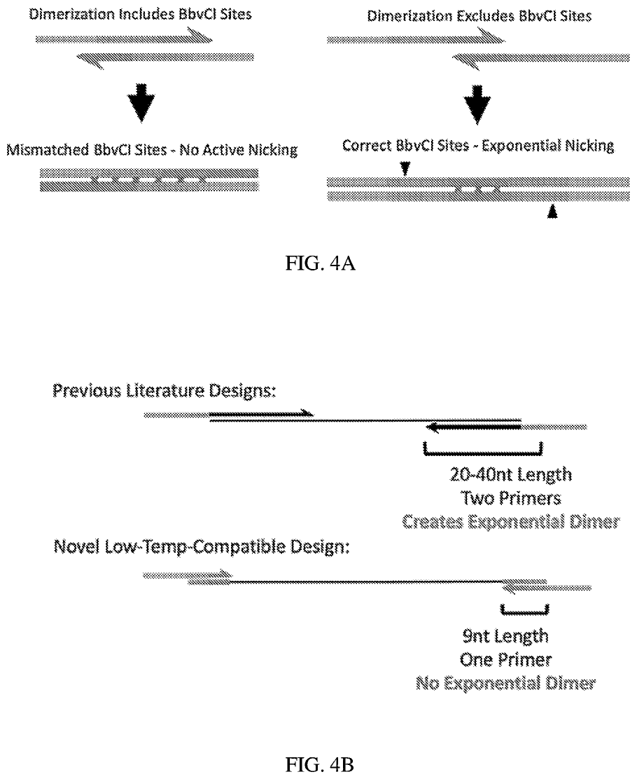

Contrary to claims by Zhou et al., the CRISDA platform does not robustly amplify and detect 220 aM of dsDNA target at 25° C. (Zhou et al. Nature Comm. vol. 9, no. 1. 2018, Supplementary FIG. 4). In attempts to replicate this single experiment, the effective limit of detection of a ssDNA target turned out to be approximately three orders of magnitude worse than claimed, despite optimization of both primer concentration and single-stranded binding protein concentration (FIG. 2).

### Example 2: Strand Displacement Amplification for Low-Temperature Use

The previous strand-displacement amplification (SDA) reaction relies on a three-step process starting from a dsDNA product containing opposing nickase recognition sequences. First, a nickase cleaves at a target sequence, leaving a free 3′ OH end available. Second, a strand-displacing DNA polymerase elongates from the freed 3′ OH and displaces the top strand of the dsDNA duplex from the nicked sequence onward. Third, a primer binds in the reverse complementary direction to a nickase recognition sequence, and the DNA polymerase elongates the primer, producing a copy of the initial dsDNA. In this fashion the dsDNA is exponentially amplified, with each nicking and elongation event producing a ssDNA copy that can then be made double-stranded again. However, this SDA reaction does not robustly perform at low temperatures (e.g., below about 25° C.).

The failure mode of SDA reactions at lower temperatures was not immediately apparent. It was initially suspected that the reaction speeds of the two enzymes involved had slowed too much to provide efficient amplification (a nickase and a strand-displacing polymerase, Nb.BbvCI and Klenow LF, respectively). However, upon assessing the rate of nicking and strand displacement carried out at room temperature compared with higher temperatures, this alone could not explain the dramatic reduction in sensitivity observed. Instead, somewhat reduced reaction kinetics were exacerbating the degree to which primer dimer products would form before sufficient levels of copies of the target polynucleotide could be generated (FIG. 3). While in theory the single-stranded binding protein (SSB, T4gp32) in the reaction is meant to prevent this by artificially raising the melting temperature of nucleic acids, it was observed that higher concentrations of SSB protein did not seem to improve the limit of detection (FIG. 3).

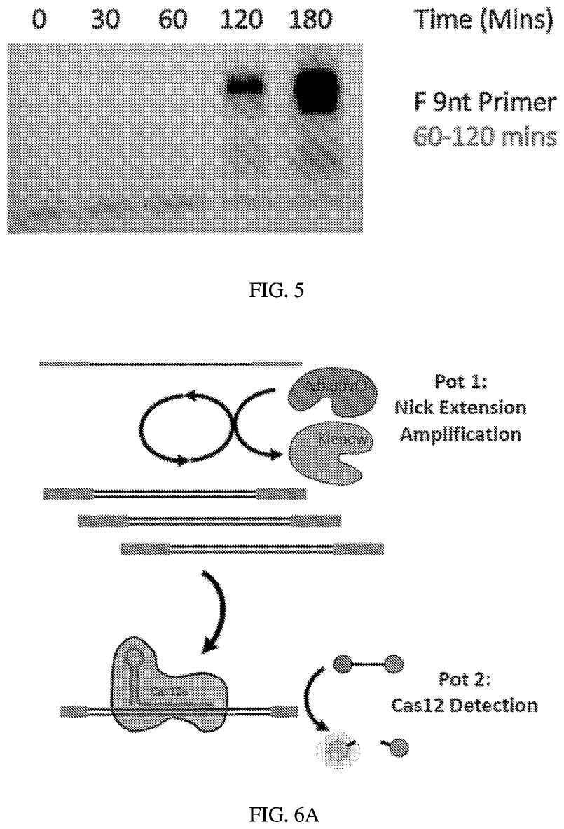

While at higher temperatures (typically 37-55° C., Zhou et al. Nature Comm. 2018 and Biolabs, New England. “Isothermal Amplification & Strand Displacement.”) primer dimer formation is of lesser consequence, it seemed likely to be the key bottleneck to lower-temperature performance of the reaction. In an initial design iteration, it was hypothesized that a short primer length would eliminate from the primer dimer pool species containing correctly paired nickase recognition sequences that might induce further exponential amplification of the dimer. The standard design for a pair of forwards and reverse SDA primers necessitates their length being in excess of 14 nt, and typically in the range of 20-40 nt (FIG. 4) (Zhou et al. Nature Comm. 2018, Biolabs, New England. “Isothermal Amplification & Strand Displacement.” And Shi et al. Analytical Chemistry, vol. 86, no. 1, 2013, pp. 336-339). Using a novel short single-primer design (where both forwards and reverse binding sequences are identical) with only 9 nt of hybridization sequence, experiments showed only linear and no exponential primer dimer formation (FIG. 5).

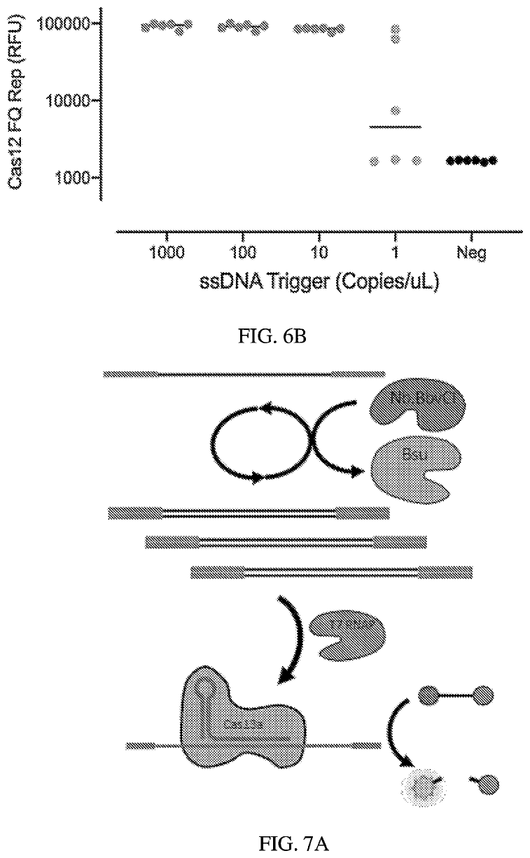

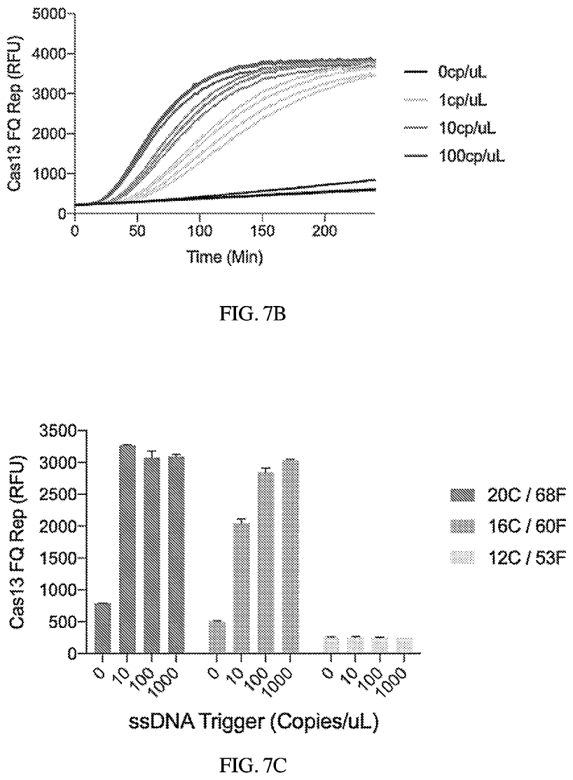

Using this optimized primer, up to single-digit copies of a ssDNA target were detectable within a two-hour reaction time using Cas12a as an amplicon detector (FIG. 6A-B). This low-attomolar LOD (˜20 aM) achieved at ambient temperature surpassed the CRISDA platform both as the authors originally reported and particularly as carried here. A further improvement was made by swapping out LbaCas12a for LwaCas13a as the amplicon detector. The inclusion of T7 RNAP and rNTPs in the reaction, as well as a T7 promoter sequence on the 5′ end of the amplification primer, allowed for the SDA amplification and Cas13-detection steps to be collapsed into a one pot for real-time detection of ssDNA trigger (FIG. 7A. The resulting assay consistently achieved detection at a concentration of 1 cp/μL (˜2 aM) (FIGS. 7B-7C).

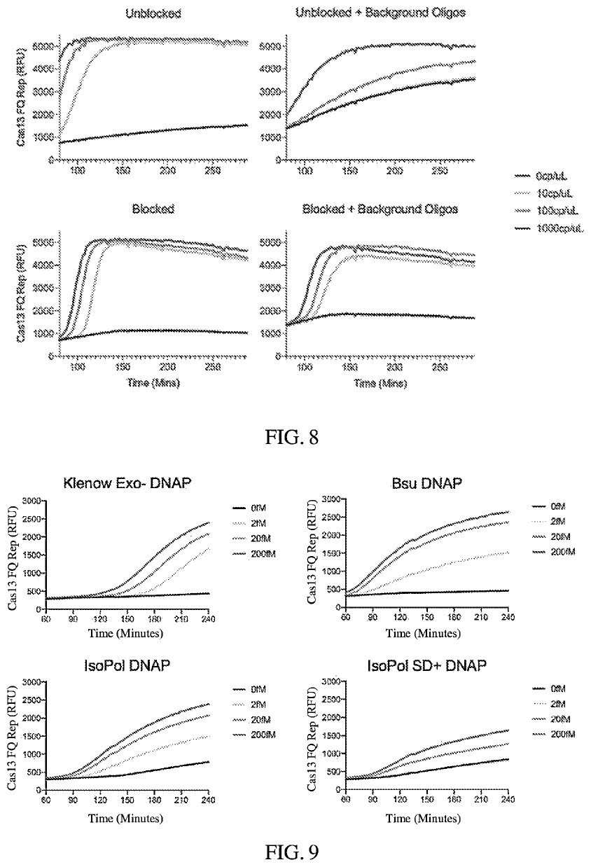

Despite the dramatic improvement in sensitivity delivered by novel short primers, there was further opportunity to optimize the system. Importantly, primer dimer continued to form, though more slowly and in the form of inert non-nicking species. While this did not prevent the detection of ssDNA targets, it did reduce the robustness of the system, in particular when adding other “background” ssDNA oligos (FIG. 8). The cause of this inhibitory interaction was not fully understood, but in general it was hypothesized that the continued ability for the short primer to be nonspecifically elongated was leading to off-target processes exacerbated by the presence of “fouling/background” free 3′ ends, even if the primer dimer formed was not exponentially amplifying. Again, this highlights the difficulty of performing isothermal amplification at low temperatures.

To eliminate this obstacle, a further novel iteration of the short primer was engineered. In this design a 3′ hexanediol blocking molecule was added to the short primers. This blocking group prevented the primer from being extended on its own by the polymerase, such that neither exponential nor non-exponential off-target products can form. The 3′ blocking end does not prevent nicking by the nickase. Upon being nicked, a 3′ end is internally liberated within the primer, which can be elongated by the DNA Polymerase, effectively unblocking the primer. Therefore, subsequent to the initial nicking, a normal double-stranded amplicon is generated that is capable of subsequent rounds of nicking and strand displacement despite the primer having initially been blocked. As shown in FIG. 8, this results in a system capable of amplifying low copy numbers of ssDNA target even in the presence of “fouling/background” oligos, dramatically improving robustness.

The challenge of adapting isothermal amplification technologies to lower ambient temperatures has largely gone unaddressed since their first design iterations over two decades ago. Here, it is shown that while lower catalytic activity of enzymes at ambient temperature compared to 37-55° C. temperature range may have a deleterious effect on amplification, a greater bottleneck for sustaining amplification at such temperatures is the production of primer dimer. By ameliorating this through novel primer design, it has been shown that amplification can be sustained even at low temperatures with limits of detection approaching single-digit copy number.

### Example 3: Amplifying Target Polynucleotides Using Different Strand Displacing Polymerases

Different polymerases can be used to amplify femtomolar (fM) amount of target polynucleotide to detectable levels. DNA Polymerases (DNAPs) tested were Klenow Exo-DNAP. Bsu DNAP, IsoPol DNAP, and IsoPol SD+DNAP. Starting target polynucleotide concentration was 0 fM, 2 fM, 20 fM, and 200 fM. Detection was performed using a Cas13a fluorophore quencher (FQ) reporter. Results showed that each polymerase was able to amplify fM concentrations of target polynucleotide sufficient to be detected. Klenow Exo-DNAP, Bsu DNAP, and IsoPol DNAP amplifications were sufficient to detect 2 fM of starting target polynucleotide after about 90 to 120 minutes of incubation. See FIG. 9.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited. In the claims, as well as in the specification above, all transitional phrases such as “comprising.” “including.” “carrying.” “having.” 5 “containing.” “involving.” “holding.” “composed of.” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between and including the upper and lower ends of the range are specifically contemplated and described herein.

What is claimed is:

1. A composition comprising:
(a) a target polynucleotide comprising a primer binding sequence, wherein the primer binding sequence comprises a nickase recognition sequence; and
(b) a primer comprising a hybridization sequence complementary to the primer binding sequence, wherein (i) the primer further comprises a 3′ blocking molecule and/or (ii) the hybridization sequence consists of 8-12 contiguous nucleotides complementary to the primer binding sequence.

2. The composition of claim 1, wherein the target polynucleotide is single stranded.

3. The composition of claim 1 or 2, wherein the nickase recognition sequence is a partial or complete nickase recognition sequence.

4. The composition of any one of the preceding claims, wherein the primer binding sequence is in a 5′ end region of the target polynucleotide.

5. The composition of any one of the preceding claims, wherein the target polynucleotide comprises an additional sequence that is a reverse complement to the primer binding sequence.

6. The composition of claim 5, wherein the additional sequence is in a 3′ end region of the target polynucleotide.

7. The composition of any one of the preceding claims, wherein the 3′ blocking molecule is selected from 3′ hexanediol, 3′ddC, 3′ Inverted dT, 3′ carbon chain spacer, 3′ amino, and 3′ phosphorylation.

8. The composition of any one of the preceding claims, wherein the primer further comprises a stabilization sequence 5′ to the nickase recognition sequence, optionally wherein the stabilization sequence consists of 6-30 nucleotides or 18 nucleotides.

9. The composition of any one of the preceding claims, wherein the target polynucleotide is present in the composition at a concentration of less than 100 attomolar.

10. The composition of any one of the preceding claims, further comprising a cognate nickase, a single-strand binding protein, a strand displacing polymerase, or any combination thereof.

11. The composition of claim 10, wherein the cognate nickase is selected from Nb.BbvCI, Nt.BspQI, Nt.CviPII, Nt.BstNBI, Nb.BsrDI, Nb.BtsI, Nt. AlwI, Nt.BbvCI, Nb.BsmI, Nb.BssSI, and Nt.BsmAI.

12. The composition of claim 10 or 11, wherein the single-strand binding protein is selected from T4 Gene 32 Protein (T4gp32), Tth RecA, and ET SSB.

13. The composition of any one of claims 10-12, wherein the strand displacing polymerase is selected from Bsu DNA Polymerase I (Bsu), phi29, Bst DNA Polymerase, Klenow Large Fragment, Klenow Exo-, Bsu Large Fragment, Isopol, and Isopol SD+.

14. The composition of any one of the preceding claims, further comprising (i) a Cas13a or Cas 13b protein, (ii) a detector polynucleotide comprising a sequence flanked by a detectable molecule and a quencher molecule, (iii) a crRNA, and (iv) an RNA polymerase, wherein the target polynucleotide further comprises a cognate RNA polymerase binding sequence, optionally the crRNA comprises a sequence that binds to RNA transcribed from the target polynucleotide.

15. A method comprising incubating the composition of claim 14 in a buffer to produce multiple copies of the target polynucleotide.

16. The method of claim 15, wherein the incubating is between about 4 degrees Celsius to about 30 degrees Celsius, or about 16 degrees Celsius to about 25 degrees Celsius.

17. The method of claim 15 or 16, wherein the incubating is in isothermal conditions.

18. The method of any one of claims 15-17, further comprising detecting the multiple copies of the target polynucleotide, optionally wherein the detecting uses specific high-sensitivity enzymatic reporter unlocking (SHERLOCK).

19. A method comprising incubating the composition of claim 13 in a buffer to produce multiple copies of the target polynucleotide.

20. The method of claim 19, wherein the incubating is between about 4 degrees Celsius to about 30 degrees Celsius, or about 16 degrees Celsius to about 25 degrees Celsius.

21. The method of claim 19 or 20, wherein the incubating is in isothermal conditions.

22. The method of any one of claims 19-21, further comprising incubating the multiple copies of the target polynucleotide with (i) a Cas13a or Cas13b protein, (ii) a detector polynucleotide comprising a sequence flanked by a detectable molecule and a quencher molecule, (iii) a crRNA, and (iv) an RNA polymerase, wherein the target polynucleotide further comprises a cognate RNA polymerase binding sequence, and wherein the crRNA comprises a sequence that binds to RNA transcribed from the target polynucleotide.

23. The method of claim 22, further comprising detecting the multiple copies of the target polynucleotide, optionally wherein the detecting uses specific high-sensitivity enzymatic reporter unlocking (SHERLOCK).

24. A method comprising incubating the composition of claim 13 in a buffer to produce multiple copies of the target polynucleotide.

25. The method of claim 24, wherein the incubating is between about 4 degrees Celsius to about 30 degrees Celsius, or about 16 degrees Celsius to about 25 degrees Celsius.

26. The method of claim 24 or 25, wherein the incubating is in isothermal conditions.

27. The method of any one of claims 24-26, further comprising incubating the multiple copies of the target polynucleotide with a Cas12a protein, a crRNA, and a detector polynucleotide comprising a detector sequence flanked by a detectable molecule and a quencher molecule, wherein the crRNA comprises a sequence that binds to the target polynucleotide or the reverse complement of the target polynucleotide.

28. The method of claim 27, further comprising detecting the multiple copies of the target polynucleotide, optionally wherein the detecting uses specific high-sensitivity enzymatic reporter unlocking (SHERLOCK).


SEQUENCE LISTING (7 sequences)

SEQ ID NO: 1 - DNA, 10 bp, synthetic construct
  source 1..10: mol_type = other DNA
ctcctcctca

SEQ ID NO: 2 - DNA, 10 bp, synthetic construct
  source 1..10: mol_type = other DNA
tgaggaggag

SEQ ID NO: 3 - DNA, 16 bp, synthetic construct
  source 1..16: mol_type = other DNA
gaaggtcgaagatcgc

SEQ ID NO: 4 - DNA, 26 bp, synthetic construct
  source 1..26: mol_type = other DNA
gaaggtcgaagatcgctgaggaggag

SEQ ID NO: 5 - DNA, 12 bp, synthetic construct
  source 1..12: mol_type = other DNA
cgtgaggaggag

SEQ ID NO: 6 - DNA, 13 bp, synthetic construct
  source 1..13: mol_type = other DNA
tgaggaggagnnn

SEQ ID NO: 7 - DNA, 13 bp, synthetic construct
  source 1..13: mol_type = other DNA
nnnctcctcctca